(19) United States
(12) Patent Application Publication
(10) Pub. No.: US 2022/0161006 A1
(43) Pub. Date: May 26, 2022

(54) PERIPHERAL ARTERY BALLOON, A HYPO-TUBE FOR PREPARING A PERIPHERAL ARTERY BALLOON AND METHODS OF USE THEREOF

(71) Applicants: Jihad Ali MUSTAPHA, Grandville, MI (US); Ali Jihad MUSTAPHA, Grandville, MI (US)

(72) Inventors: Jihad Ali MUSTAPHA, Grandville, MI (US); Ali Jihad MUSTAPHA, Grandville, MI (US)

(21) Appl. No.: 17/533,782

(22) Filed: Nov. 23, 2021

Related U.S. Application Data
(60) Provisional application No. 63/118,215, filed on Nov. 25, 2020.

(51) Int. Cl. A61M 25/10

(52) U.S. Cl. CPC A61M 25/104; A61M 25/1002

(57) ABSTRACT

A peripheral artery balloon is provided that includes a distal end having a first outer diameter, a proximal end having a second outer diameter that is larger than the first outer diameter, and an extended portion that extends from the distal end to the proximal end, the extended portion having an outer diameter that gradually increases from the first outer diameter of the distal end to the second outer diameter of the proximal end. The distal end, proximal end, and extended portion of the peripheral artery balloon are sized to mimic a varying diameter of a natural artery. A hypo-tube for preparing a peripheral artery balloon is also provided, as well as a method of remodeling and/or treating arteries after an atherectomy and/or during an angioplasty using a peripheral artery balloon.

### CROSS-REFERENCE TO RELATED APPLICATIONS

This application claims priority to U.S. Provisional Application No. 63/118,215 filed Nov. 25, 2020, the entire contents of which is hereby incorporated by reference.

### TECHNICAL FIELD

The present invention relates to a peripheral artery balloon for peripheral vascular remodeling after an interventional procedure (e.g., an atherectomy and/or balloon angioplasty), along with a hypo-tube for preparing a peripheral artery balloon. The peripheral artery balloon and/or the hypo-tube for preparing the peripheral artery balloon has a tapered wall thickness and/or a change in the outer diameter of the balloon and/or the hypo-tube in order to mimic the natural shape of native peripheral arteries when remodeling these arteries after, for example, an atherectomy and/or during an angioplasty.

### BACKGROUND

Peripheral artery disease (PAD) is a common manifestation of systemic atherosclerosis causing a chronic, slowly developing, narrowing of the arteries in the legs and feet. Lower extremity peripheral artery disease (LE-PAD) typically affects lower limbs with a frequency that is strongly age-related (˜20% of the population is older than 55). Atherosclerosis affecting lower limbs has some specific features: diffuse involvement, superimposed calcification, high incidence of progression to total occlusion, and/or large plaque burden. Various interventional procedures, such as atherectomy and/or balloon angioplasty, exist to address and/or treat these conditions.

There remains a need for an improved procedure to remodel and/or repair the affected arteries of the upper leg (above the knee) and lower leg (below the knee) after an interventional procedure, such as an atherectomy and/or balloon angioplasty.

### BRIEF SUMMARY OF THE INVENTION

According to one embodiment, the present invention provides a peripheral artery balloon that includes a distal end having a first outer diameter, a proximal end having a second outer diameter that is larger than the first outer diameter, and an extended portion that extends from the distal end to the proximal end, the extended portion having an outer diameter that gradually increases from the first outer diameter of the distal end to the second outer diameter of the proximal end, wherein the distal end, proximal end, and extended portion of the peripheral artery balloon are sized to mimic a varying diameter of a natural artery.

According to an embodiment, the peripheral artery balloon further includes an inner lumen that extends from the distal end to the proximal end of the balloon. According to an embodiment, the inner lumen includes at least one port for infusion and/or deflation of the balloon.

According to one embodiment, the first outer diameter at the distal end of the balloon is 2.5 mm. According to an embodiment, the second outer diameter at the proximal end of the balloon is 4 mm.

According to one embodiment, the extended portion includes at least two transitions in an outer diameter of the balloon between the distal end and the proximal end of the balloon. According to an embodiment, a first transition of the extended portion has an outer diameter that is 0.5 mm larger than the first outer diameter at the distal end of the balloon. According to an embodiment, a second transition of the extended portion has an outer diameter that is 0.5 mm larger than the outer diameter at the first transition of the extended portion. According to an embodiment, the second outer diameter at the proximal end of the balloon is 0.5 mm larger than the outer diameter at the second transition of the extended portion. According to an embodiment, (i) the first outer diameter at the distal end of the balloon is 2.5 mm, (ii) the outer diameter at the first transition of the extended portion is 3 mm, (iii) the outer diameter at the second transition of the extended portion is 3.5 mm, and (iv) the second outer diameter at the proximal end of the balloon is 4 mm.

According to one embodiment, the balloon is non-compliant (high pressure). According to one embodiment, the balloon is semi-complaint (mid-pressure). According to one embodiment, the balloon is compliant (elastomeric).

According to one embodiment, the natural artery is the popliteal artery. According to another embodiment, the natural artery is the superficial femoral artery (SFA).

According to an embodiment, the first outer diameter of the distal end, the second outer diameter of the proximal end, and the gradually increasing outer diameter of the extended portion mimics the varying diameter of a natural artery in a 1:1 ratio.

According to one embodiment, the present invention provides a hypo-tube for preparing a peripheral artery balloon that includes a distal end having a first outer diameter, a proximal end having a second outer diameter that is larger than the first outer diameter, and an extended portion that extends from the distal end to the proximal end, the extended portion having an outer diameter that gradually increases from the first outer diameter of the distal end to the second outer diameter of the proximal end, wherein the distal end, proximal end, and extended portion of the hypo-tube are sized to prepare a peripheral artery balloon that mimics a varying diameter of a natural artery.

According to an embodiment, the hypo-tube further includes an inner lumen that extends from the distal end to the proximal end of the hypo-tube. According to one embodiment, the inner lumen has a substantially constant outer diameter that is the same size from the distal end to the proximal end of the hypo-tube. According to another embodiment, the inner lumen has an outer diameter that gradually increases in size from the distal end to the proximal end of the hypo-tube. According to another embodiment, an outer diameter of the inner lumen at the proximal end of the hypo-tube is 0.1 mm larger than the outer diameter of the inner lumen at the distal end of the hypo-tube.

According to an embodiment, a thickness of the hypo-tube is defined between an outer diameter of an inner lumen and the outer diameter of the hypo-tube According to one embodiment, a thickness at the distal end of the hypo-tube is the same as a thickness at the proximal end of the hypo-tube. According to another embodiment, a thickness (α) at the distal end of the hypo-tube is less than a thickness (β) at the proximal end of the hypo-tube. According to one embodiment, a thickness (α) at the distal end of the hypo-tube is 0.5 mm and a thickness (β) at the proximal end of the hypo-tube is 1 mm. According to an embodiment, the thickness gradually increases from the distal end of the hypo-tube to the proximal end of the hypo-tube.

According to one embodiment, the first outer diameter at the distal end of the hypo-tube is 2.5 mm. According to an embodiment, the second outer diameter at the proximal end of the hypo-tube is 4 mm.

According to one embodiment, the extended portion includes at least two transitions in an outer diameter of the hypo-tube between the distal end and the proximal end of the hypo-tube. According to an embodiment, a first transition of the extended portion has an outer diameter that is 0.5 mm larger than the first outer diameter at the distal end of the hypo-tube. According to an embodiment, a second transition of the extended portion has an outer diameter that is 0.5 mm larger than the outer diameter at the first transition of the extended portion. According to an embodiment, the second outer diameter at the proximal end of the hypo-tube is 0.5 mm larger than the outer diameter at the second transition of the extended portion. According to an embodiment, (i) the first outer diameter at the distal end of the hypo-tube is 2.5 mm, (ii) the outer diameter at the first transition of the extended portion is 3 mm, (iii) the outer diameter at the second transition of the extended portion is 3.5 mm, and (iv) the second outer diameter at the proximal end of the hypo-tube is 4 mm.

According to one embodiment, the outer diameter of the extended portion of the hypo-tube that gradually increases from the first outer diameter of the distal end to the second outer diameter of the proximal end comprises a continuous slope. According to an embodiment, the outer diameter of the extended portion of the hypo-tube is free of step changes in diameter.

According to an embodiment, the first outer diameter of the distal end, the second outer diameter of the proximal end, and the gradually increasing outer diameter of the extended portion creates a peripheral artery balloon that mimics the varying diameter of a natural artery in a 1:1 ratio.

According to another embodiment, the present invention provides a method of remodeling arteries of a patient after an atherectomy. The method includes (a) providing a single peripheral artery balloon, the single peripheral artery balloon having an outer diameter that varies from a distal end to a proximal end of the balloon in a manner that mimics a varying diameter of a natural artery, (b) inserting the single peripheral artery balloon into a damaged artery of the patient, (c) expanding the single peripheral artery balloon to remodel the damaged artery, (d) deflating the single peripheral artery balloon, and (e) removing the single peripheral artery balloon from the patient.

According to one embodiment, the single peripheral artery balloon is configured to remodel the popliteal artery of the leg without any other balloons being inserted into the popliteal artery of the leg. According to another embodiment, the single peripheral artery balloon is configured to remodel the superficial femoral artery (SFA) of the leg without any other balloons being inserted into the SFA of the leg.

According to yet another embodiment, the present invention provides a method of treating arteries of a patient. The method includes (a) providing a single peripheral artery balloon, the single peripheral artery balloon having an outer diameter that varies from a distal end to a proximal end of the balloon in a manner that mimics a varying diameter of a natural artery, (b) inserting the single peripheral artery balloon into an artery of the patient, (c) expanding the single peripheral artery balloon to treat the artery, (d) deflating the single peripheral artery balloon, and (e) removing the single peripheral artery balloon from the patient.

According to one embodiment, the single peripheral artery balloon is configured to treat the popliteal artery of the leg without any other balloons being inserted into the popliteal artery of the leg. According to another embodiment, the single peripheral artery balloon is configured to treat the superficial femoral artery (SFA) of the leg without any other balloons being inserted into the SFA of the leg.

According to an embodiment, the method comprises a balloon angioplasty.

Additional features, advantages, and embodiments of the invention are set forth or apparent from consideration of the following detailed description, drawings and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1A is an illustration of the basic anatomy for the arteries of the lower leg, including the popliteal artery.

FIG. 1B is an illustration of the basic anatomy for the arteries of the upper leg, including the superficial femoral artery (SFA).

FIG. 2 is a diagram of the placement of three balloons in the popliteal artery of the lower leg during a procedure of remodeling the artery after an atherectomy and/or during an angioplasty.

FIG. 3 illustrates a hypo-tube for preparing a peripheral artery balloon according to an embodiment of the invention.

FIG. 4A illustrates a portion of a hypo-tube for preparing a peripheral artery balloon according to an embodiment of the invention.

FIGS. 4B and 4C illustrate cross-sectional views of the hypo-tube for preparing a peripheral artery balloon of FIG. 4A taken along sections 4B-4B and 4C-4C, respectively, of the hypo-tube.

FIG. 5 illustrates a portion of a hypo-tube for preparing a peripheral artery balloon according to an embodiment of the invention.

FIGS. 6A-6C illustrate cross-sectional views of the hypo-tube for preparing a peripheral artery balloon of FIG. 5 taken along sections 6A-6A, 6B-6B, and 6C-6C, respectively, of the balloon.

FIG. 7 illustrates a hypo-tube for preparing a peripheral artery balloon having a large step change in the outer diameter of the hypo-tube.

FIG. 8 illustrates a hypo-tube for preparing a peripheral artery balloon according to an embodiment of the invention.

FIGS. 9A-9E illustrate various hypo-tubes for preparing peripheral artery balloons according embodiments of the invention.

FIG. 10 illustrates a portion of hypo-tube before and after becoming a peripheral artery balloon according to an embodiment of the invention.

FIG. 11 illustrates a hypo-tube that becomes a balloon having a plurality of infusion and/or deflation ports according to an embodiment of the invention.

FIG. 12A illustrates a peripheral artery balloon in an inflated state according to an embodiment of the invention.

FIG. 12B illustrates a peripheral artery balloon in a semi-inflated state according to an embodiment of the invention.

FIG. 12C illustrates a peripheral artery balloon in a deflated state according to an embodiment of the invention.

FIG. 13 is a diagram of the popliteal artery at the knee joint that can be treated during a procedure of remodeling the artery after an atherectomy and/or during an angioplasty.

FIG. 14A is a diagram of the popliteal artery at the knee joint having a peripheral artery balloon inserted therein in order to perform a procedure of remodeling the artery after an atherectomy and/or during an angioplasty.

FIG. 14B illustrates the peripheral artery balloon that is inserted into the popliteal artery of FIG. 14A.

### DETAILED DESCRIPTION OF THE INVENTION

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though they may. Furthermore, the phrases “in another embodiment” and “in some other embodiments” as used herein do not necessarily refer to a different embodiment, although they may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, terms such as “comprising,” “including,” and “having” do not limit the scope of a specific claim to the materials or steps recited by the claim.

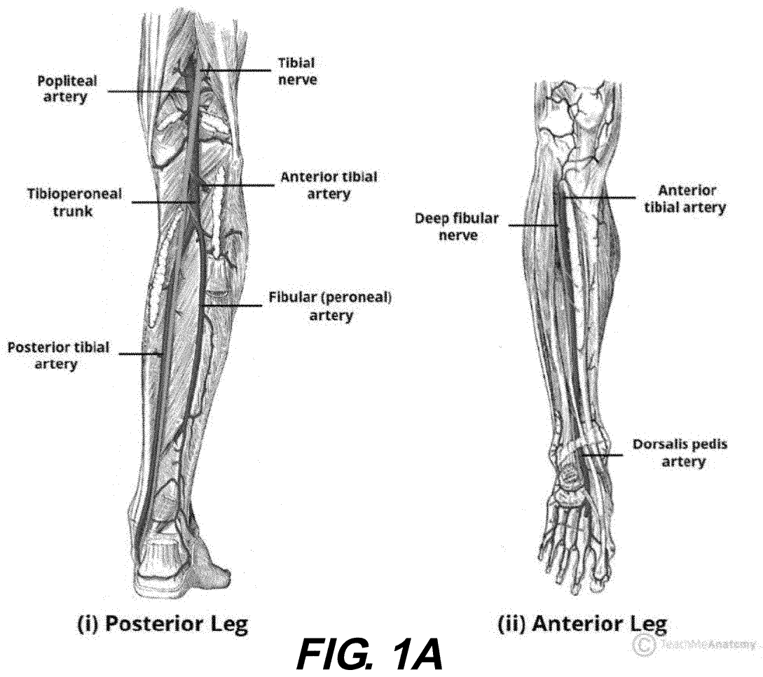

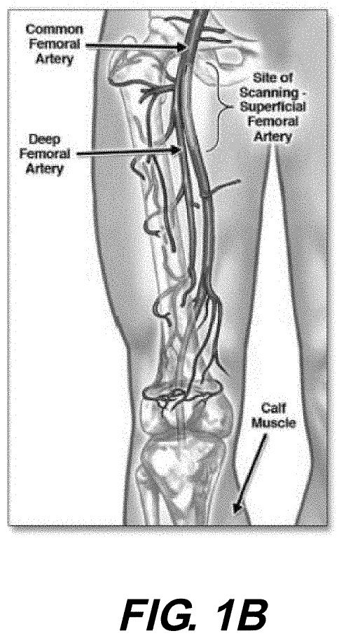

One method to treat PAD is the use of atherectomy. An atherectomy is a procedure to remove plaque build-up (atherosclerosis) that is narrowing or blocking one or more of the arteries. During an atherectomy, fatty buildup (i.e., plaque) is cut away and removed to widen the artery and improve blood flow. When an atherectomy is used to treat the arteries of, for example, the legs and feet, the narrow arteries of the legs and feet can be damaged due to this invasive procedure (see, e.g., FIGS. 1A and 1B). Thus, methods are used to remodel the damaged arteries including, e.g., the use of peripheral artery balloons to repair and/or remodel the impacted peripheral arteries.

Another method to treat PAD is balloon angioplasty. Balloon angioplasty is a procedure used to open narrowed or blocked arteries. It uses a balloon attached to a catheter that's inserted into an artery. At the place where deposits of plaque have closed off or narrowed the channel for blood flow, the balloon is inflated.

As shown in FIG. 1A, the arteries of the lower leg (e.g., below the knee) include the popliteal artery, which descends down the posterior thigh, giving rise to genicular branches that supply the knee joint. The popliteal artery divides into the anterior tibial artery and the tibioperoneal trunk. In turn, the tibioperoneal trunk bifurcates into the posterior tibial and fibular arteries, which include the posterior tibial artery and the fibular (peroneal) artery. The posterior tibial artery continues inferiorly, along the surface of the deep posterior leg muscles (such as the tibialis posterior). The posterior tibial artery enters the sole of the foot via the tarsal tunnel, accompanying the tibial nerve. The fibular (peroneal) artery descends posteriorly to the fibula, within the posterior compartment of the leg. The fibular (peroneal) artery gives rise to perforating branches, which penetrate the intermuscular septum to supply muscles in the lateral compartment of the leg. The other division of the popliteal artery, the anterior tibial artery, passes anteriorly between the tibia and fibula, through a gap in the interosseous membrane. It then moves inferiorly down the leg and runs down the entire length of the leg, and into the foot, where it becomes the dorsalis pedis artery. Thus, in order to treat the various arteries below the knee, the various branches of the popliteal artery are involved.

By contrast, the arteries of the upper leg (e.g., above the knee) include the superficial femoral artery (SFA), which is a continuation of the common femoral artery (see, e.g., FIG. 1B). The SFA is the main artery of the upper leg and is, therefore, critical in the supply of oxygenated blood to the leg.

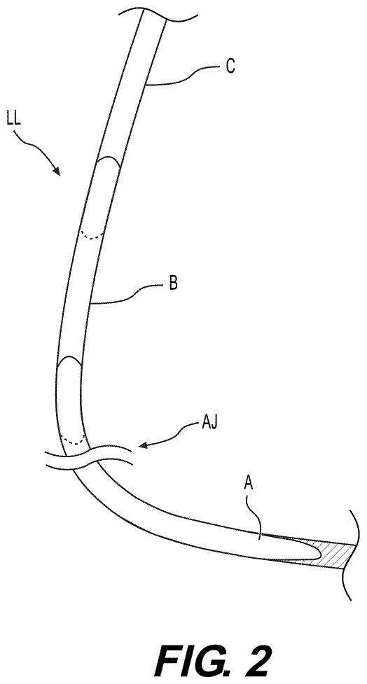

Currently, available balloons on the market have only a single outer diameter. These balloons will have to be used in a sequential fashion by using, for example, a 2.5 mm balloon first. This requires many technical steps to achieve the objective goal of balloon angioplasty and/or to treat the arteries of the leg (i.e., to treat PAD) after an atherectomy. Generally, at least two to three balloons of differing sizes are needed to perform balloon angioplasty and/or to remodel and/or repair the affected arteries of both the upper leg (above the knee) and lower leg (below the knee). For example, as shown in FIG. 2, to treat the arteries of the lower leg (LL) (i.e., below the knee), three balloons (i.e., balloons A, B, and C) are individually inserted, inflated, deflated, and removed from separate portions of the popliteal artery to perform balloon angioplasty and/or to remodel and/or repair the various arteries of the lower leg (LL) (i.e., below the knee) after an atherectomy. As shown in FIG. 2, in a first step, after having a wire track across a target lesion, a first balloon (balloon A) is first inserted into the portion of the popliteal artery that is below the ankle joint (AJ). This first balloon (balloon A) has a first size and diameter that is sized to remodel this narrow portion of the popliteal artery that is below the ankle joint (AJ) (e.g., balloon A has a single outer diameter of 2.5 mm). After inserting the first balloon (balloon A) and confirming its location is accurately at the target lesion using, for example, digital subtraction angiography (DSA), this balloon is inflated to a desired atmospheric pressure to perform balloon angioplasty and/or to remodel and/or repair this portion of the popliteal artery. This balloon is generally inflated within the target lesion for about two (2) minutes. This balloon must then be deflated (e.g., until all contrast solution from the balloon is removed using, for example, an inflation device, such as, e.g., a PRESTO® or ENCORE™ inflation device) and removed before the next portion of the popliteal artery is remodeled and/or repaired. Thereafter, the same process is repeated for a second balloon (balloon B) and a third balloon (balloon C). For example, a second balloon (balloon B) is inserted into the portion of the popliteal artery that is just above the ankle joint (AJ). This second balloon (balloon B), which is generally bigger than the first balloon (balloon A), has a second size and diameter that is sized to remodel this portion of the popliteal artery that is just above the ankle joint (AJ) (e.g., balloon B has a single outer diameter of 3 mm). After inserting the second balloon (balloon B) and confirming its location, this balloon is inflated to remodel and/or repair this portion of the popliteal artery. This balloon must then be deflated and removed before the next portion of the popliteal artery is remodeled and/or repaired. Finally, a third balloon (balloon C) is inserted into the portion of the popliteal artery that is further above the ankle joint (AJ). This third balloon (balloon C), which is generally bigger than both the first balloon (balloon A) and the second balloon (balloon B), has a third size and diameter that is sized to remodel this portion of the popliteal artery that is further above the ankle joint (AJ) (e.g., balloon C has a single outer diameter of 4 mm). After inserting the third balloon (balloon C) and confirming its location, this balloon is inflated to remodel and/or repair this portion of the popliteal artery. This balloon must then be deflated and removed.

Similarly, to remodel and/or repair the affected arteries of the upper leg (above the knee) during a balloon angioplasty and/or after an atherectomy, at least two to three balloons of differing sizes are needed to remodel and/or repair the various portions of the SFA. Accordingly, at least five to six balloons of differing sizes are used in a procedure to remodel and/or repair the affected arteries of the upper leg (above the knee) and lower leg (below the knee) during a balloon angioplasty and/or after an atherectomy, because this concept of repeated sequential balloon angioplasty and/or remodeling continues until the target artery or arteries are treated. Typically, lesions, including those that include high grade stenosis and/or are occluded, in the arteries vary in length. This means a lesion can range in length from 5 mm to 400 mm and vary in diameter from 1.5 mm to 5 mm. These lesions can also be found within the arteries below the knee and into the ankle. The same range of blockage can happen in arteries above the knee, with lesion lengths varying from 5 mm to 700 mm in length and varying in diameter from 4 mm to 7 mm. Thus, typically, numerous balloons of varying sizes (given that each balloon has a single sized outer diameter) must be used to treat the varying lengths and diameters of the lesions within the impacted arteries. Moreover, this procedure, which involves inserting, inflating, deflating, and removing each of these multiple balloons, can take between 20 to 30 minutes of time due to all of the exchanges of the various balloons. When working with various patients, including elderly patients, the more time needed to perform such a procedure while under anesthesia, the higher likelihood of various complications.

The present invention relates to a peripheral artery balloon for peripheral vascular remodeling after an interventional procedure, such as an atherectomy and/or balloon angioplasty, along with a hypo-tube for preparing a peripheral artery balloon. The peripheral artery balloon and/or the hypo-tube for preparing the peripheral artery balloon has a tapered wall thickness and/or change in the outer diameter of the balloon and/or the hypo-tube in order to mimic the natural shape of native peripheral arteries when remodeling these arteries after, for example, an atherectomy and/or during an angioplasty.

Accordingly, one embodiment includes a peripheral artery balloon that includes a distal end having a first outer diameter, a proximal end having a second outer diameter that is larger than the first outer diameter, and an extended portion that extends from the distal end to the proximal end, the extended portion having an outer diameter that gradually increases from the first outer diameter of the distal end to the second outer diameter of the proximal end, wherein the distal end, proximal end, and extended portion of the peripheral artery balloon are sized to mimic a varying diameter of a natural artery.

Another embodiment of the invention relates to a hypo-tube for preparing a peripheral artery balloon that includes a distal end having a first outer diameter, a proximal end having a second outer diameter that is larger than the first outer diameter, and an extended portion that extends from the distal end to the proximal end, the extended portion having an outer diameter that gradually increases from the first outer diameter of the distal end to the second outer diameter of the proximal end, wherein the distal end, proximal end, and extended portion of the hypo-tube are sized to prepare a peripheral artery balloon that mimics a varying diameter of a natural artery.

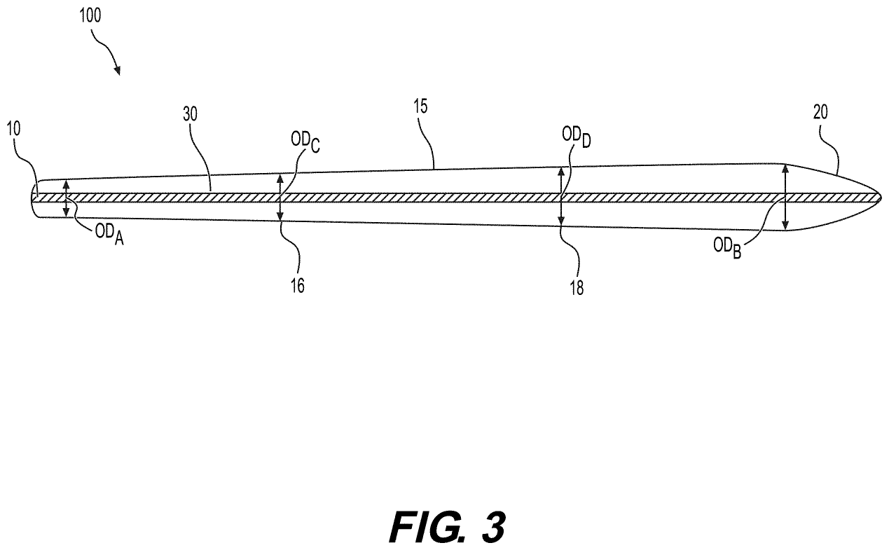

FIG. 3 illustrates a hypo-tube for preparing a peripheral artery balloon according to one embodiment of the invention. As shown in FIG. 3, the hypo-tube (100) includes a distal end (10), a proximal end (20), and an extended portion (15) the extends from the distal end (10) to the proximal end (20). The distal end (10) of the hypo-tube (10) has a first outer diameter (OD_{A}) and the proximal end (20) of the hypo-tube (10) has a second outer diameter (OD_{B}), with the second outer diameter (OD_{B}) of the proximal end (20) being larger than the first outer diameter (OD_{A}) of the distal end (10). According to the embodiment of FIG. 3, the first outer diameter (OD_{A}) is 2.5 mm, while the second outer diameter (OD_{B}) is 4 mm.

As further shown in FIG. 3, the extended portion (15) has an outer diameter that gradually increases from the first outer diameter (OD_{A}) of the distal end (10) to the second outer diameter (OD_{B}) of the proximal end (20). The extended portion (15) further includes a first transition (16) and a second transition (18). The first transition (16) includes an outer diameter (OD_{C}) and the second transition (18) includes an outer diameter (OD_{D}). According to the embodiment of FIG. 3, the outer diameter (OD_{C}) of the first transition (16) is 0.5 mm larger than the first outer diameter (OD_{A}) at the distal end (10) of the hypo-tube (100) (e.g., the outer diameter (OD_{C}) at the first transition (16) is 3 mm, while the first outer diameter (OD_{A}) at the distal end (10) is 2.5 mm). In addition, according to this embodiment, the outer diameter (OD_{D}) of the second transition (18) is 0.5 mm larger than the outer diameter (OD_{C}) at the first transition (16) (e.g., the outer diameter (OD_{C}) at the first transition (16) is 3 mm, while the outer diameter (OD_{D}) at the second transition (18) is 3.5 mm). Moreover, according to this embodiment, the second outer diameter (OD_{B}) at the proximal end (20) of the hypo-tube (100) is 0.5 mm larger than the outer diameter (OD_{D}) at the second transition (18) (e.g., the outer diameter (OD_{D}) at the second transition (18) is 3.5 mm, while the second outer diameter (OD_{B}) at the proximal end (20) is 4 mm). Thus, as shown in the embodiment of FIG. 3, the outer diameter of the hypo-tube (100) varies and gradually increases from the distal end (10) to the proximal end (20) of the hypo-tube (100) in order to prepare a peripheral artery balloon having an outer diameter that also varies and gradually increases from a distal end to a proximal end of the balloon. According to one embodiment, the first outer diameter of the distal end (e.g., OD_{A} of distal end (10)), the second outer diameter of the proximal end (e.g., OD_{B} of proximal end (20)), and the gradually increasing outer diameter of the extended portion (e.g., outer diameter (OD_{C}) and outer diameter (OD_{D})) creates a peripheral artery balloon that mimics the varying diameter of a natural artery in a 1:1 ratio.

According to one embodiment, the hypo-tube (100) for preparing a peripheral artery balloon has an overall length from the distal end (10) to the proximal end (20) of about 210 mm. A hypo-tube (100) having a length of about 210 mm allows for creating a peripheral artery balloon of a similar size that can reshape the popliteal artery of the leg, which has generally this same length. According to another embodiment, the hypo-tube (100) for preparing a peripheral artery balloon has an overall length from the distal end (10) to the proximal end (20) of about 300 mm. A hypo-tube (100) having a length of about 300 mm allows for creating a peripheral artery balloon of a similar size that can reshape the SFA of the leg, which has generally a length of about 320 mm to 350 mm.

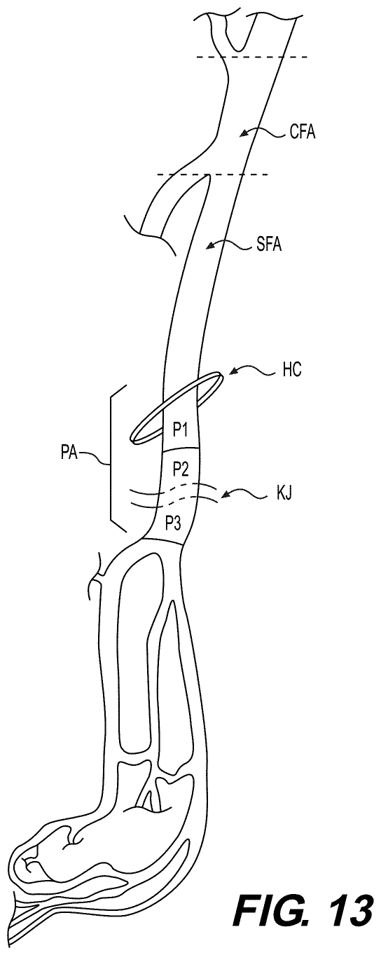

According to one embodiment, the hypo-tube can be configured to prepare a peripheral artery balloon that includes an outer diameter at one end of the balloon that is about 5 mm, which matches to a natural transition area that occurs at the area of the knee joint (KJ) between the SFA and the popliteal artery (PA) (see, e.g., “Hunter's Canal” (HC) shown in FIG. 13). This transition area or zone (e.g., regions P1-P3 of the popliteal artery (PA) of FIG. 13) can be effectively treated by creating (i) a first multi-diameter balloon that is sized to treat the popliteal artery (PA) of the leg but having this one end that is matched to this transition area/zone (e.g., regions P1-P3 of the popliteal artery (PA) of FIG. 13), and (ii) a second multi-diameter balloon that is sized to treat the SFA and/or the common femoral artery (CFA) of the leg but also having this one end that is matched to this transition area/zone (e.g., regions P1-P3 of the popliteal artery (PA) of FIG. 13).

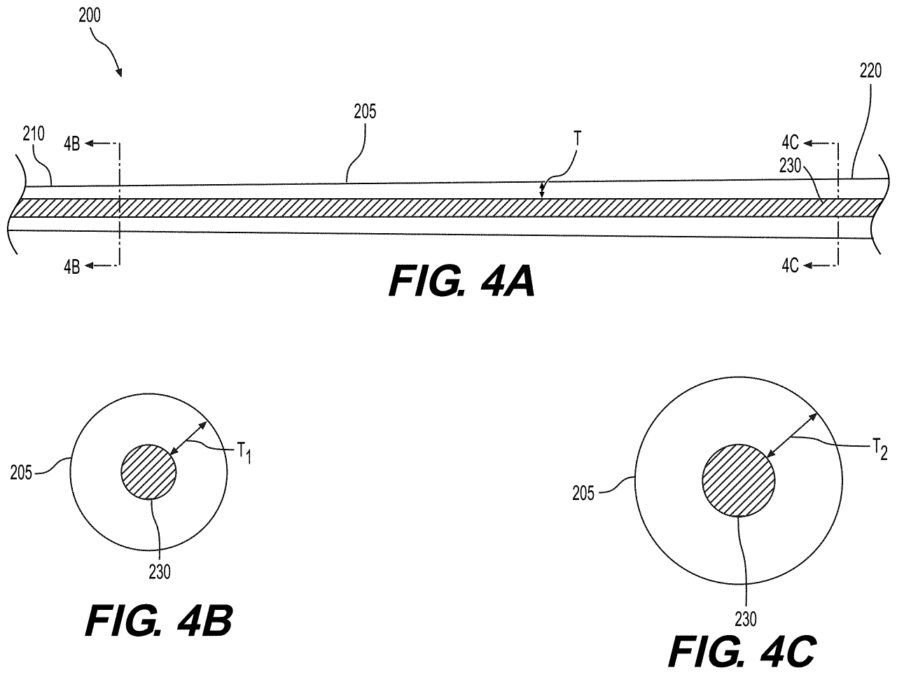

As further shown in the embodiment of FIG. 3, the hypo-tube (100) for preparing a peripheral artery balloon includes an inner lumen (30) that runs from the distal end (10) to the proximal end (20) of the hypo-tube (100). As shown in FIG. 3, the inner lumen (30) can have a diameter that is slightly larger (e.g., 0.1 mm larger) at the proximal end (20) as compared to the diameter of the inner lumen (30) at the distal end (10) of the hypo-tube. Alternatively, as shown in the embodiment of FIGS. 4A-4C, a hypo-tube (200) for preparing a peripheral artery balloon can include an inner lumen (230) that has a substantially constant diameter that is the same size from a distal end (210) to a proximal end (220) of the hypo-tube (200).

As further shown in the embodiment of FIGS. 4A-4C, a thickness (T) is defined between the inner lumen (230) and the external wall (205) of the hypo-tube (200). The thickness (T) will be (i) a first thickness (T₁) at the distal end (210) of the hypo-tube (200) and (ii) a second thickness (T₂) at the proximal end (220) of the hypo-tube (200). According to one embodiment, the first thickness (T₁) will be 0.5 mm at the distal end (210) of the hypo-tube (200), and the second thickness (T₂) will be 1 mm at the proximal end (220) of the hypo-tube (200). Thus, as shown in the embodiment of FIGS. 4A-4C, the thickness (T) of the hypo-tube (200) varies and gradually/sequentially increases from the distal end (210) to the proximal end (220) of the hypo-tube (200) to thus prepare a peripheral artery balloon that also varies and gradually/sequentially increases from the distal end to the proximal end of the balloon.

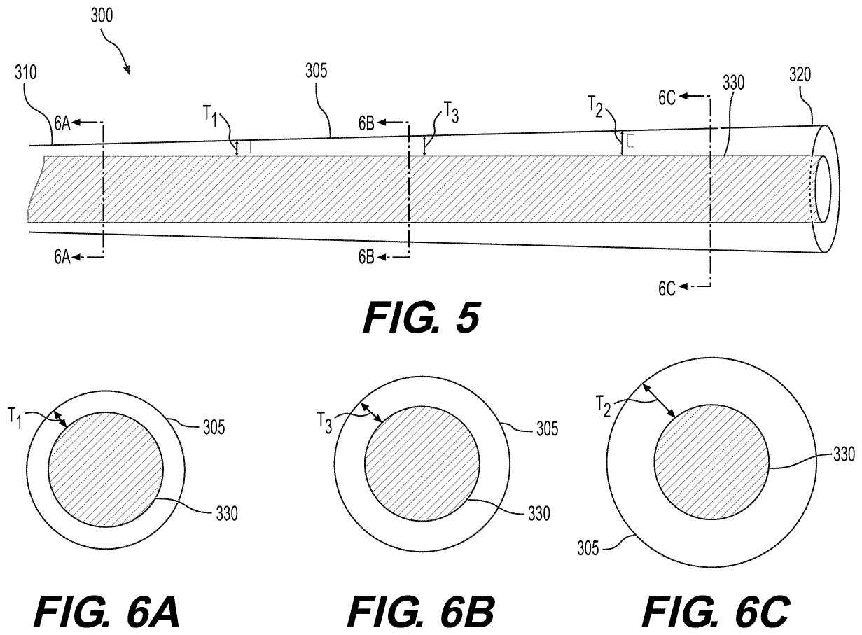

FIG. 5 illustrates another embodiment of a hypo-tube (300) for preparing a peripheral artery balloon, the hypo-tube (300) having a thickness that gradually increases from a distal end (310) to a proximal end (320) of the hypo-tube (300) (see, e.g., thicknesses T₁, T₂, and T₃). As in the embodiment of FIGS. 4A-4C, a thickness (T₁, T₂, and/or T₃) of the hypo-tube (300) of FIG. 5 is defined between an inner lumen (330) and the external wall (305) of the hypo-tube (300). As shown in the embodiment of FIG. 5, a first thickness (α)(T₁) is defined at the distal end (310) of the hypo-tube (300), which is less than a second thickness (β)(T₂) at the proximal end (320) of the hypo-tube (300). According to an embodiment, the thickness (α) at the distal end of the hypo-tube is 0.5 mm and the thickness (β) at the proximal end of the hypo-tube is 1 mm. According to the embodiment of FIG. 5, the inner lumen (330) is consistent in its diameter from the distal end (310) to the proximal end (320) of the hypo-tube (300). The outer diameter for this hypo-tube (300), however, is variable. According to one embodiment, the outer diameter for the hypo-tube (300) is determined by starting at the tip, e.g., the distal end (310), of the hypo-tube (300). This distal end (310) or tip will have a very thin wall thickness (e.g., thickness (α)(T₁)), which gradually increases from the distal end (310) to the proximal end (320) of the hypo-tube (300) (see, e.g., FIGS. 6A-6C), to thus prepare a peripheral artery balloon that gradually increases in diameter from a distal end to a proximal end of the balloon.

FIGS. 6A-6C illustrate the gradual increase in the thickness of the hypo-tube (300) from the distal end (310) to the proximal end (320) of the hypo-tube (300) of FIG. 5. As shown in FIG. 6A, the distal end (310) of the hypo-tube has a first thickness (α)(T₁) that is defined between the inner lumen (330) and the external wall (305) of the hypo-tube (300). FIG. 6C illustrates the proximal end (320) of the hypo-tube (300), which has a second thickness (β)(T₂) that is defined between the inner lumen (330) and the external wall (305) of the hypo-tube (300). According to this embodiment, the second thickness (β)(T₂) at the proximal end (320) is larger than the first thickness (α)(T₁) at the distal end (310) of the hypo-tube (300). FIG. 6B illustrates a portion of the hypo-tube (300) that is between the distal end (310) and the proximal end (320) of the hypo-tube (300), which has a third thickness (T₃) that is defined between the inner lumen (330) and the external wall (305) of the hypo-tube (300). According to this embodiment, the third thickness (T₃) is greater than the first thickness (α)(T₁) at the distal end (310) of the hypo-tube (300), however, the third thickness (T₃) is less than the second thickness (β)(T₂) at the proximal end (320) of the hypo-tube (300). Accordingly, as shown in the embodiments of FIGS. 5-6C, the thickness (T₁, T₂, and T₃) of the hypo-tube (300) varies and gradually/sequentially increases from the distal end (310) to the proximal end (320) of the hypo-tube (300), to thus prepare a peripheral artery balloon with a diameter that varies and gradually/sequentially increases from the distal end to the proximal end of the balloon.

According to one embodiment, the thickness of the hypo-tube varies from a thickness at the distal end (i.e., first thickness (α)(T₁)) to a thickness at the proximal end (i.e., second thickness (β)(T₂)). The difference in thickness between the thickness at the distal end (i.e., first thickness (α)(T₁)) and the thickness at the proximal end (i.e., second thickness (β)(T₂)) can be a principal contribution toward the ability to develop a multi-diameter balloon that will have the same compliance as the conventionally used balloons, despite the variance/difference in the diameter of the balloon of the present invention. Moreover, this difference in the thickness of the hypo-tube can be configured to relate to the wall thickness of the target lesion, which also varies from the proximal end to the distal end of the lesion. Therefore, using a multi-diameter balloon according to embodiments of the invention is crucial for the patient to have the best outcome.

According to an embodiment, the wall thickness of the hypo-tube at the distal end (i.e., first thickness (α)(T₁)) is pre-set to form a balloon size with an outer diameter that expands to the maximum pre-set compliance creating an outer diameter with a standard deviation of −0.3 to +0.3 based on the total atmospheric pressure delivered into the balloon via an inflation device. If the atmospheric pressure exceeds the pre-set nominal pressure, then the outer diameter of the balloon will expand to an additional outer diameter of +0.3 above the pre-set outer diameter size. In this case, any additional atmospheric pressure increase could cause maximal intra-balloon pressure with potential balloon wall weakness. In other words, if the balloon reaches a certain atmospheric pressure, with all of the other elements combined, the balloon could rupture within the vessel.

According to one embodiment, an inner tubing and/or a hypo-tube for manufacturing a multi-diameter balloon of the present invention has the same size inner lumen/diameter. According to an embodiment, an outer diameter of tubing (e.g., hypo-tube) for manufacturing a multi-diameter balloon of the present invention varies in its outer diameter. According to one embodiment, the distal tip (or end) of the tubing has a proportionally smaller outer diameter as compared to the proximal end/portion of the same tubing.

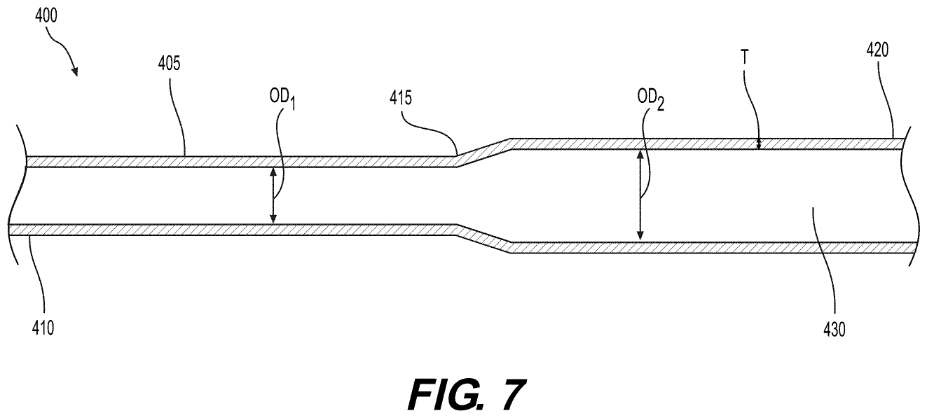

FIG. 7 illustrates a hypo-tube (400) having a distal end (410) and a proximal end (420). The hypo-tube (400) of FIG. 7 further has an inner lumen (430) that includes a first outer diameter (OD₁) at the distal end (410) of the hypo-tube (400) and a second outer diameter (OD₂) at the proximal end (420) of the hypo-tube (400). As shown in the embodiment of FIG. 7, the first outer diameter (OD₁) is smaller at the distal end (410) of the hypo-tube (400) than the second outer diameter (OD₂) at the proximal end (420) of the hypo-tube (400). A thickness (T) is further defined between the inner lumen (430) and the external surface (405) of the hypo-tube (400). However, according to this embodiment, the thickness (T) is constant. Thus, as the thickness (T) remains constant, the change in size of the first outer diameter (OD₁) to the second outer diameter (OD₂) causes the hypo-tube, and thus, a peripheral artery balloon prepared therefrom to increase in size and/or diameter from the distal end (410) to the proximal end (420) of the hypo-tube (400). This increase in the size of the first outer diameter (OD) to the second outer diameter (OD₂) is not gradual. Thus, a sharp transition or step (415) occurs between the distal end (410) and the proximal end (420) of the hypo-tube (400). Such a sharp transition or step (415) in the hypo-tube (400) is not preferred because this can result in a prepared balloon that can cause complications within the artery as the balloon is expanded or inflated. For example, such a sharp transition or step (415) can cause a dissection or tearing of the artery during the expansion or inflation of a balloon prepared from this hypo-tube (400).

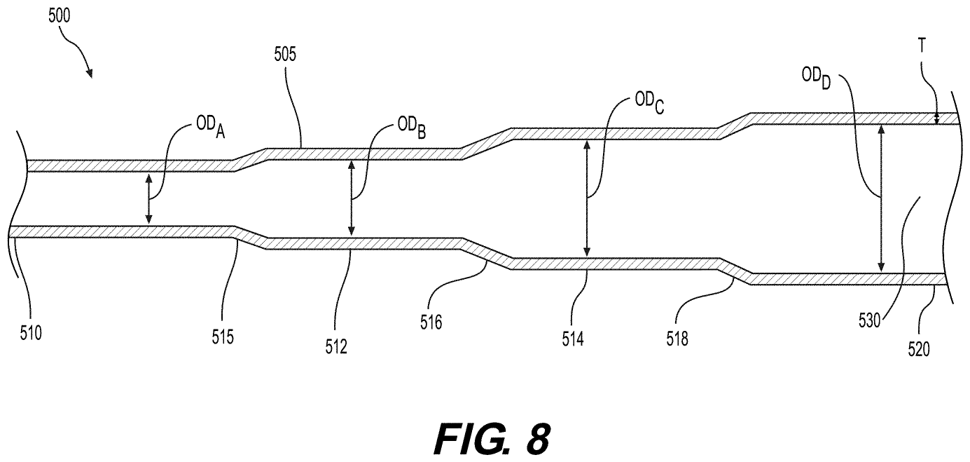

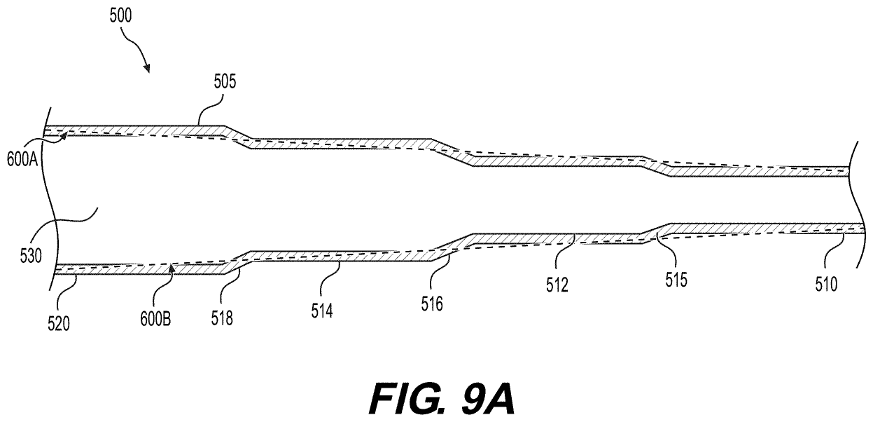

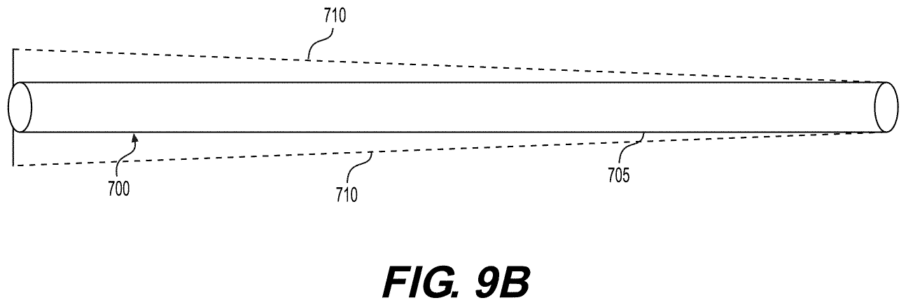

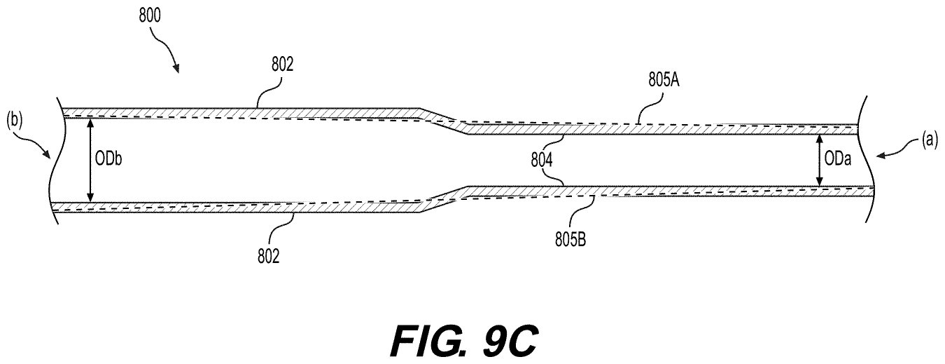

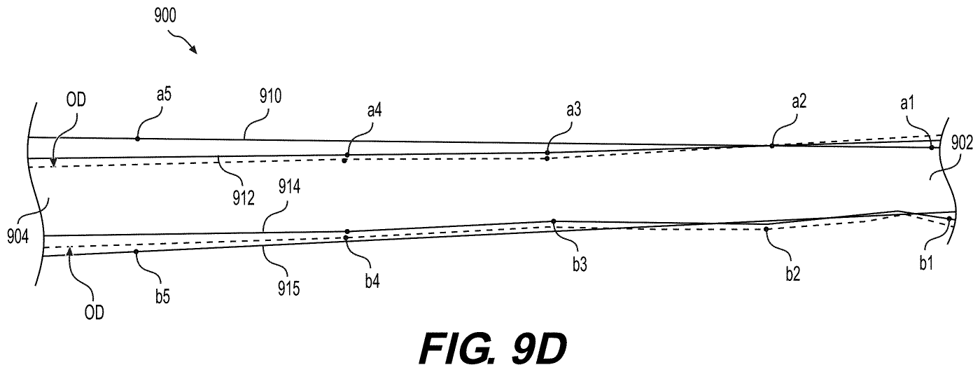

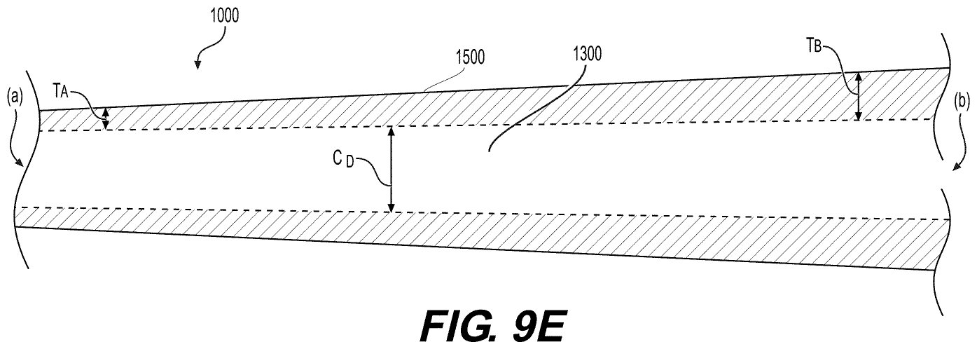

By contrast, FIG. 8 illustrates a hypo-tube (500) having a gradual increase in the size or diameter of the hypo-tube (500) from a distal end (510) to a proximal end (520) of the hypo-tube (500). For example, the hypo-tube (500) of FIG. 8 has an inner lumen (530) that includes (i) a first outer diameter (OD_{A}) at the distal end (510) of the hypo-tube (500), (ii) a second outer diameter (OD_{B}) that is larger than the first outer diameter (OD_{A}) at a first portion (512) of the hypo-tube (500), (iii) a third outer diameter (OD_{C}) that is larger than both the second outer diameter (OD_{B}) and the first outer diameter (OD_{A}) at a second portion (514) of the hypo-tube (500), and (iv) a fourth outer diameter (OD_{D}) at the proximal end (520) of the hypo-tube (500), with the fourth outer diameter (OD_{D}) being larger than each of the third outer diameter (OD_{C}), the second outer diameter (OD_{B}), and the first outer diameter (OD_{A}). As further shown in the embodiment of FIG. 8, a thickness (T) is defined between the inner lumen (530) and the external surface (505) of the hypo-tube (500). Similar to the embodiment of FIG. 7, the thickness (T) is constant. Thus, as the thickness (T) remains constant, the change in size of the outer diameter of the inner lumen (530) from the first outer diameter (OD_{A}) to the second outer diameter (OD_{B}) to the third outer diameter (OD_{C}) and finally to the fourth outer diameter (OD_{D}) causes the hypo-tube, and thus, a peripheral artery balloon prepared therefrom, to increase in size from the distal end (510) to the proximal end (520) of the hypo-tube (500). However, according to the embodiment of FIG. 8, the increase in the size of the outer diameter of the inner lumen (530) is gradual (e.g., has a continuous slope). Accordingly, a gradual transition occurs between each change in the outer diameter of the inner lumen (530). In particular, as shown in the embodiment of FIG. 8, a first gradual transition (515) occurs between the distal end (510) (having the first outer diameter (OD_{A})) and the first portion (512) of the hypo-tube (500) having the second outer diameter (OD_{B}). Thereafter, a second gradual transition (516) occurs between the first portion (512) of the hypo-tube (500) having the second outer diameter (OD_{B}) and the second portion (514) of the hypo-tube (500) having the third outer diameter (OD_{C}). Finally, a third gradual transition (518) occurs between the second portion (514) of the hypo-tube (500) having the third outer diameter (OD_{C}) and the proximal end (520) of the hypo-tube (500) (having the fourth outer diameter (OD_{D})). These gradual transitions (515, 516, 518) avoid the sharp transition or steps shown in the embodiment of FIG. 7 (see, e.g., transition/step (415) of FIG. 7), which thus, avoids various complications that can occur within the artery as a balloon prepared from such a hypo-tube is expanded or inflated. For example, as shown in FIGS. 9A-9E, according to the embodiment of FIG. 8, which is repeated in the embodiment of FIG. 9A, an average line/slope (600A, 600B) angled from the distal end (510) to the proximal end (520) shows a precise, sequential, incremental pattern. Thus, the average summation (710) of this angled line/slope (600A, 600B) of FIG. 9A will display a shallow angle (705) for the balloon or tubing (700) (see, e.g., FIG. 9B). In addition, as shown in FIG. 9C, the average sequential diameter increases for the hypo-tube (800) are created by using a line (805A, 805B) drawn between (i) the outer lining (802) of the tubing at the distal end (a) and (ii) the inner lining (804) of tubing at the proximal end (b). Moreover, as shown in FIG. 9C, the summation of the average lines (805A, 805B) from the distal end (a) to proximal end (b) of the hypo-tube show significant separation between the balloon or tube's outer diameter (ODa) at the distal end (a) as compared to the balloon or tube's outer diameter (ODb) at the proximal end (b). In addition, as shown in FIG. 9D, significant variations in the outer diameter (OD) are illustrated at the different segments (a1 to a5 and b1 to b5) of the hypo-tube (900) from the distal end (902) to the proximal end (904). In particular, there is a spontaneous increase and/or decrease in the outer diameter (OD) of the hypo-tube (900) as shown by the location of origin of different summation points (e.g., outer line 910 to inner line 912 (at, e.g., segments a1 to a5) and inner line 914 to outer line 915 (at, e.g., segments b1 to b5)). FIG. 9E illustrates yet another embodiment of a hypo-tube (1000) having a gradual increase in the size or diameter of the hypo-tube (1000) from a distal end (a) to a proximal end (b) of the hypo-tube (1000). For example, the hypo-tube (1000) of FIG. 9E has an inner lumen (1300) that includes a constant outer diameter (CD) from the distal end (a) of the hypo-tube (1000) to the proximal end (b) of the hypo-tube (1000). As further shown in the embodiment of FIG. 9E, a thickness (T_{A}) is defined between the inner lumen (1300) and an external surface (1500) of the hypo-tube (1000) at the distal end (a) of the hypo-tube (1000), while a thickness (T_{B}) is defined between the inner lumen (1300) and the external surface (1500) of the hypo-tube (1000) at the proximal end (b) of the hypo-tube (1000). Similar to the embodiment of FIG. 5, the thickness varies from the distal end (a) to the proximal end (b) of the hypo-tube (1000), such that the thickness (T_{A}) at the distal end (a) of the hypo-tube (1000) is less than the thickness (T_{B}) at the proximal end (b) of the hypo-tube (1000). Thus, as the thickness changes from the distal end (a) of hypo-tube (1000) to the proximal end (b) of the hypo-tube (1000), i.e., the thickness increases from T_{A} to T_{B}, the outer diameter of the hypo-tube (1000) also increases, and thus, a peripheral artery balloon prepared therefrom will increase in size from the distal end (a) to the proximal end (b) of the hypo-tube (1000).

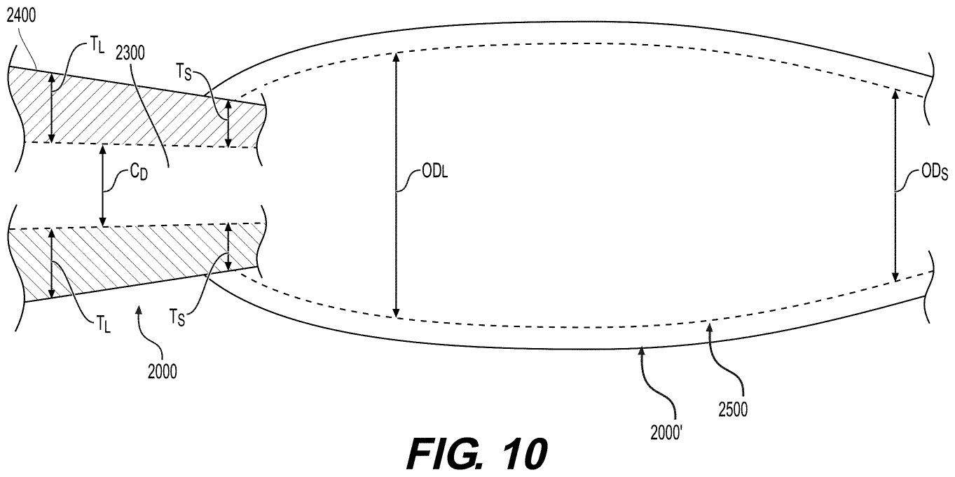

FIG. 10 illustrates a portion of hypo-tube before and after becoming a peripheral artery balloon according to an embodiment of the invention. As shown in FIG. 10, a portion of a hypo-tube (2000) is illustrated, along with a portion of hypo-tube (2000′) that is prepared into and/or becomes a peripheral artery balloon (2500). Similar to the hypo-tube (1000) of FIG. 9E, the hypo-tube (2000) of FIG. 10 has an inner lumen (2300) that includes a constant outer diameter (CD) from a distal end (not shown) of the hypo-tube (2000) to a proximal end (not shown) of the hypo-tube (2000). As further shown in the embodiment of FIG. 10, a thickness (T_{L}) is defined between the inner lumen (2300) and an external surface (2400) of the hypo-tube (2000) at a portion of the hypo-tube (2000), while a thickness (T_{S}) is defined between the inner lumen (2300) and the external surface (2400) of the hypo-tube (2000) at another portion of the hypo-tube (2000). Similar to the embodiment of FIG. 9E, the thickness varies along the various portions of the hypo-tube (2000), such that the thickness (T_{L}) is greater than the thickness (T_{S}). As the thickness changes along the various portions of the hypo-tube (2000), i.e., the thickness decreases from T_{L} to T_{S}, the outer diameter of the hypo-tube (2000) also decreases, and thus, a peripheral artery balloon (2500) prepared therefrom will vary in size depending upon this varying thickness of the hypo-tube (2000). For example, as shown in FIG. 10, the peripheral artery balloon (2500) prepared from the hypo-tube (2000) has an outer diameter that varies, with an outer diameter (OD_{L}) of the balloon (2500) being the largest along a portion of the balloon (2500) that relates to the thickest portion of the hypo-tube (2000) from which the balloon (2500) is prepared, i.e., the portion with the largest thickness (T_{L}). The outer diameter of the peripheral artery balloon (2500) begins to taper down to an outer diameter (OD_{S}) that is less than the largest outer diameter (OD_{L}) of the balloon (2500). This tapering of the outer diameter of the balloon (2500) from the largest outer diameter (OD_{L}) to a smaller outer diameter (OD_{S}) relates to the change in thickness of the hypo-tube (2000), i.e., the thickness decrease from T_{L} to T_{S}. According to one embodiment, the variable thickness of the hypo-tube (2000) provides flexibility and room to build a balloon (e.g., peripheral artery balloon (2500)) with variable balloon diameters (see, e.g., outer diameters OD_{L} and OD_{S}). In addition, according to one embodiment, a thickness of the hypo-tube (2000) at the highest level (e.g., the thickness T_{L}) allows for building the largest diameter of a balloon (e.g., the outer diameter (OD_{L}) of the balloon (2500)), whereas the thinnest portion of the hypo-tube (2000) (e.g., the thickness T_{S}) allows for building a smaller (or smallest) diameter of a balloon (e.g., the outer diameter (OD_{S}) of the balloon (2500)).

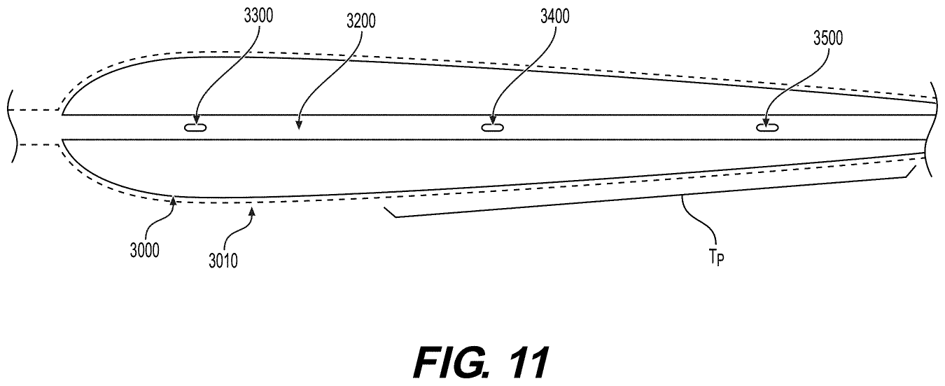

FIG. 11 illustrates a hypo-tube that becomes a balloon having a plurality of infusion and/or deflation ports according to an embodiment of the invention. In particular, as shown in FIG. 11, a hypo-tube (3010) is prepared into and/or becomes a peripheral artery balloon (3000). The peripheral artery balloon (3000) includes an inner lumen (3200) having a plurality of infusion and/or deflation ports (3300, 3400, 3500). These infusion and/or deflation ports (3300, 3400, 3500) allow for inflating and/or deflating the peripheral artery balloon (3000). In addition, as shown in FIG. 11, the peripheral artery balloon (3000) has a varying diameter, such that the peripheral artery balloon (3000) tapers along the region (Tr) of the balloon (3000) as shown.

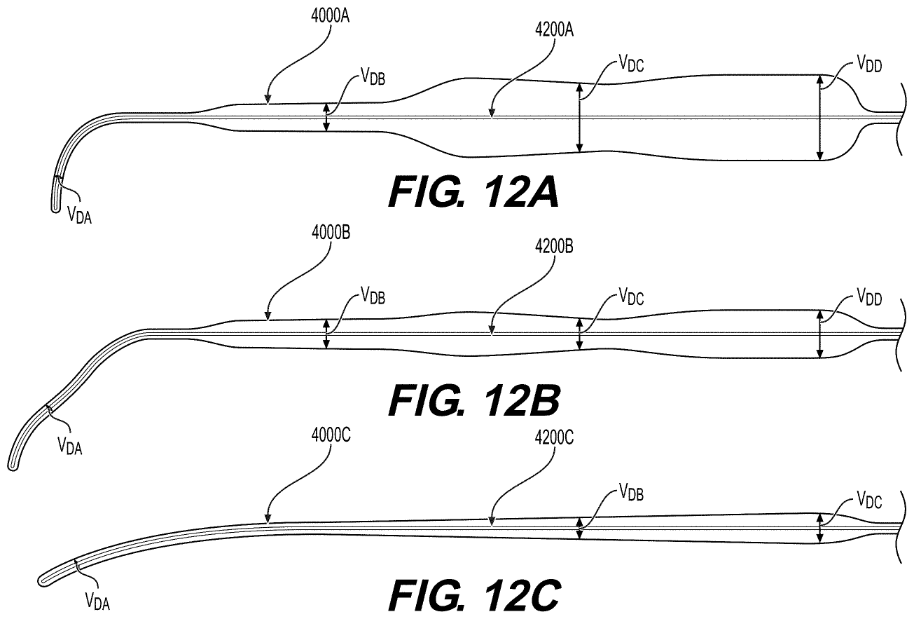

FIGS. 12A-12C illustrate a peripheral artery balloon in varying states of inflation, semi-inflation, and deflation according to an embodiment of the invention. FIG. 12A illustrates a peripheral artery balloon (4000A) that is inflated. As shown in FIG. 12A, the peripheral artery balloon (4000A) has an inner lumen (4200A) and a varying diameter. In particular, in the inflated state of FIG. 12A, the peripheral artery balloon (4000A) has at least four distinct diameters that range from a smallest diameter (VDA) to a largest diameter (VDo), with at least two different diameters (VDB and VDc) in between the smallest diameter (VDA) and the largest diameter (VDD). FIG. 12B illustrates a peripheral artery balloon (4000B) that is semi-inflated. As shown in FIG. 12B, the peripheral artery balloon (4000B) has an inner lumen (4200B) and a varying diameter. Similar to the inflated state of FIG. 12A, the peripheral artery balloon (4000B) in the semi-inflated state of FIG. 12B also has at least four distinct diameters that range from a smallest diameter (VDA) to a largest diameter (VDD), with at least two different diameters (VDe and VDc) in between the smallest diameter (VDA) and the largest diameter (VDD). FIG. 12C illustrates a peripheral artery balloon (4000C) that is deflated. As shown in FIG. 12C, the peripheral artery balloon (4000C) has an inner lumen (4200C) and a varying diameter. However, unlike the inflated state of FIG. 12A and the semi-inflated state of FIG. 12B, the diameters of the peripheral artery balloon (4000C) in the deflated state are less distinct. In particular, as shown in FIG. 12C, the peripheral artery balloon (4000C) has about three noticeable diameters that range from a smallest diameter (VDA) to a largest diameter (VDc), with at least one diameter (VDa) in between the smallest diameter (VDA) and the largest diameter (VDc). According to one embodiment, the inner lumen (4200A, 4200B, 4200C) of the peripheral artery balloons (4000A, 4000B, 4000C) of FIGS. 12A-12C can be an inflation and/or deflation tube, as well as a guide wire lumen. According to one embodiment, the balloon is non-compliant (high pressure). According to one embodiment, the balloon is semi-complaint (mid-pressure). According to one embodiment, the balloon is compliant (elastomeric).

Another embodiment includes a method of remodeling arteries of a patient after an atherectomy. The method includes (a) providing a single peripheral artery balloon, the single peripheral artery balloon having an outer diameter that varies from a distal end to a proximal end of the balloon in a manner that mimics a varying diameter of a natural artery, (b) inserting the single peripheral artery balloon into a damaged artery of the patient, (c) expanding the single peripheral artery balloon to remodel the damaged artery, (d) deflating the single peripheral artery balloon, and (e) removing the single peripheral artery balloon from the patient.

According to one embodiment, the single peripheral artery balloon is configured to remodel the popliteal artery of the leg without any other balloons being inserted into the popliteal artery of the leg. According to another embodiment, the single peripheral artery balloon is configured to remodel the superficial femoral artery (SFA) of the leg without any other balloons being inserted into the SFA of the leg.

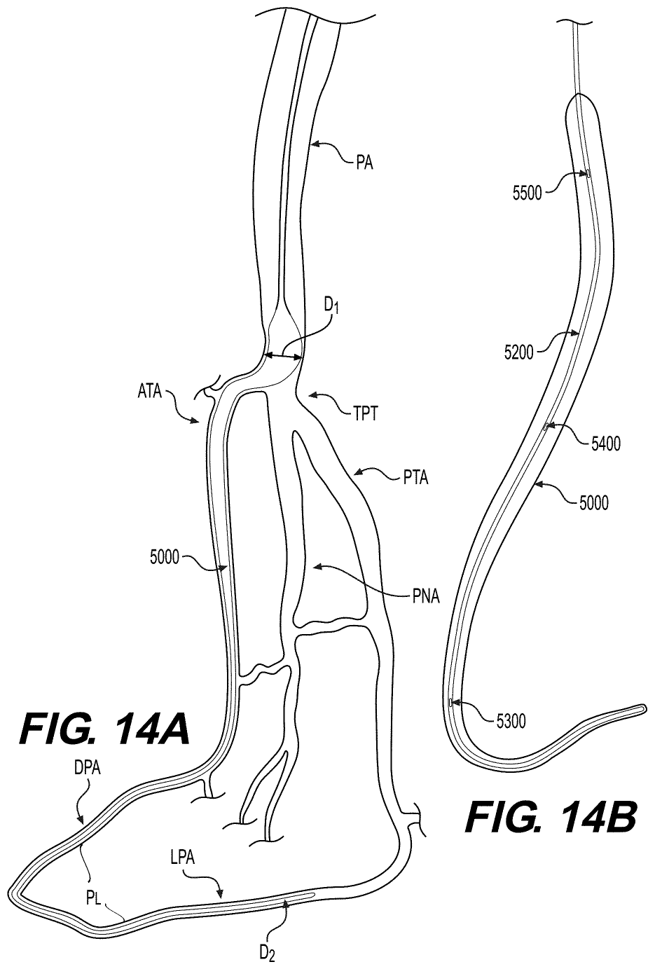

FIG. 14A illustrates a diagram of the popliteal artery (PA) at the knee joint having a single peripheral artery balloon inserted therein in order to perform a procedure of remodeling the artery after an atherectomy and/or during an angioplasty. In particular, as shown in FIG. 14A, a single peripheral artery balloon (5000) is inserted at and below the PA for treating the various portions below the PA, including, e.g., the anterior tibial artery (ATA), the dorsalis pedis artery (DPA), the pedal loop (PL), and the lateral plantar artery (LPA). The single peripheral artery balloon (5000) is a long, tapered balloon having a largest diameter (D₁) near the PA. According to one embodiment, the largest diameter (D₁) of the single peripheral artery balloon (5000) is about 4.5 mm to about 5 mm, which mimics the size of this portion of the PA. The peripheral artery balloon (5000) tapers from the largest diameter (D₁) near one end of the balloon (5000) to a smallest diameter (D₂) near the opposite end of the balloon (5000). According to one embodiment, the smallest diameter (D₂) of the single peripheral artery balloon (5000) is about 1.5 mm to about 2.5 mm, which mimics the size of the LPA. While the embodiment of FIG. 14A illustrates a single peripheral artery balloon (5000) being used to perform a procedure of remodeling and/or treating the PA and various portions below the PA, including, e.g., the anterior tibial artery (ATA), the dorsalis pedis artery (DPA), the pedal loop (PL), and the lateral plantar artery (LPA), the single peripheral artery balloon (5000) could also be used to treat the other portions below the PA, including, e.g., the tibial peroneal trunk (TPT), the posterior tibial artery (PTA), and/or the peroneal artery (PNA). Thus, according to the embodiment of FIG. 14A, a single peripheral artery balloon (5000) can be used to treat the arteries of the lower leg (LL) (i.e., below the knee), which is in contrast to the embodiment of FIG. 2 in which three balloons (i.e., balloons A, B, and C) are individually inserted, inflated, deflated, and removed from separate portions of the popliteal artery (PA) to perform balloon angioplasty and/or to remodel and/or repair the various arteries of the lower leg (LL) (i.e., below the knee) after an atherectomy.

FIG. 14B illustrates the single peripheral artery balloon (5000) that is inserted into the popliteal artery (PA) of FIG. 14A. As shown in FIG. 14B, the single peripheral artery balloon (5000) includes an inner lumen (5200), as well as a plurality of inflation and/or deflation ports (5300, 5400, 5500). According to one embodiment, the plurality of inflation and/or deflation ports (5300, 5400, 5500) allow for inflating and/or deflating the peripheral artery balloon (5000), while the balloon (5000) is inserted within the PA and the various portions below the PA, as discussed above.

Another embodiment includes a method of treating arteries of a patient. The method includes (a) providing a single peripheral artery balloon, the single peripheral artery balloon having an outer diameter that varies from a distal end to a proximal end of the balloon in a manner that mimics a varying diameter of a natural artery, (b) inserting the single peripheral artery balloon into an artery of the patient, (c) expanding the single peripheral artery balloon to treat the artery, (d) deflating the single peripheral artery balloon, and (e) removing the single peripheral artery balloon from the patient.

According to one embodiment, the single peripheral artery balloon is configured to treat the popliteal artery of the leg without any other balloons being inserted into the popliteal artery of the leg. According to another embodiment, the single peripheral artery balloon is configured to treat the superficial femoral artery (SFA) of the leg without any other balloons being inserted into the SFA of the leg.

According to an embodiment, the method comprises a balloon angioplasty.

According to embodiments of the invention, by providing (a) a single peripheral artery balloon that is configured to remodel and/or treat the popliteal artery of the leg without any other balloons being inserted into the popliteal artery of the leg and/or (b) a single peripheral artery balloon that is configured to remodel and/or treat the superficial femoral artery (SFA) of the leg without any other balloons being inserted into the SFA of the leg, there is at least a six fold reduction in potential complications due to (i) a reduction in the number of balloon exchanges (e.g., the two balloons of the invention vs. five to six balloons as currently used in these procedures) and/or (ii) a reduction in the number of inflations (e.g., the inflation of the two balloons of the invention vs. the inflation of five to six balloons as currently used in these procedures). In addition, according to an embodiment of the invention, the procedure of the instant invention that uses (a) a single peripheral artery balloon that is configured to remodel and/or treat the popliteal artery of the leg and (b) a single peripheral artery balloon that is configured to remodel and/or treat the superficial femoral artery (SFA) can be finished in about two minutes, as opposed to the 20 to 30 minutes currently required to perform these types of procedures with numerous balloons.

According to an embodiment of the invention, after inflating or expanding a peripheral artery balloon that is configured to remodel and/or treat a damaged or impacted artery according to the invention, the damaged artery will generally take the shape of the inflated/expanded peripheral artery balloon. After deflation and/or removal of the peripheral artery balloon, the remodeled artery often decreases in size, with a decrease of about 15% to 20% from the inflated/expanded peripheral artery balloon. However, by providing a peripheral artery balloon with varying diameters that mimics the varying diameter of the natural, undamaged artery (i.e., a 1.1 ratio between the varying diameter of the peripheral artery balloon and the varying diameter of the natural, undamaged artery), a more effective remodeling of the damaged tissue can occur. Moreover, according to embodiments of the invention, a high pressure balloon is possible, while preventing dissection or tearing of the damaged artery during remodeling.

Only exemplary embodiments of the present invention and but a few examples of its versatility are shown and described in the present disclosure. It is to be understood that the present invention is capable of use in various other combinations and environments and is capable of changes or modifications within the scope of the inventive concept as expressed herein.

Further aspects of the present disclosure are provided by the subject matter of the following clauses.

A peripheral artery balloon comprising a distal end having a first outer diameter; a proximal end having a second outer diameter that is larger than the first outer diameter; and an extended portion that extends from the distal end to the proximal end, the extended portion having an outer diameter that gradually increases from the first outer diameter of the distal end to the second outer diameter of the proximal end, wherein the distal end, proximal end, and extended portion of the peripheral artery balloon are sized to mimic a varying diameter of a natural artery.

The peripheral artery balloon of any preceding clause, further comprising an inner lumen that extends from the distal end to the proximal end of the balloon.

The peripheral artery balloon of any preceding clause, wherein the inner lumen includes at least one port for infusion and/or deflation of the balloon.

The peripheral artery balloon of any preceding clause, wherein the first outer diameter at the distal end of the balloon is 2.5 mm.

The peripheral artery balloon of any preceding clause, wherein the second outer diameter at the proximal end of the balloon is 4 mm.

The peripheral artery balloon of any preceding clause, wherein the extended portion includes at least two transitions in an outer diameter of the balloon between the distal end and the proximal end of the balloon.

The peripheral artery balloon of any preceding clause, wherein a first transition of the extended portion has an outer diameter that is 0.5 mm larger than the first outer diameter at the distal end of the balloon.

The peripheral artery balloon of any preceding clause, wherein a second transition of the extended portion has an outer diameter that is 0.5 mm larger than the outer diameter at the first transition of the extended portion.

The peripheral artery balloon of any preceding clause, wherein the second outer diameter at the proximal end of the balloon is 0.5 mm larger than the outer diameter at the second transition of the extended portion.

The peripheral artery balloon of any preceding clause, wherein (i) the first outer diameter at the distal end of the balloon is 2.5 mm, (ii) the outer diameter at the first transition of the extended portion is 3 mm, (iii) the outer diameter at the second transition of the extended portion is 3.5 mm, and (iv) the second outer diameter at the proximal end of the balloon is 4 mm.

The peripheral artery balloon of any preceding clause, wherein the balloon is one of (i) non-compliant, (ii) semi-complaint, or (iii) compliant.

The peripheral artery balloon of any preceding clause, wherein the natural artery is one of the popliteal artery and the superficial femoral artery (SFA).

The peripheral artery balloon of any preceding clause, wherein the first outer diameter of the distal end, the second outer diameter of the proximal end, and the gradually increasing outer diameter of the extended portion mimics the varying diameter of a natural artery in a 1:1 ratio.

A hypo-tube for preparing a peripheral artery balloon, the hypo-tube comprising a distal end having a first outer diameter; a proximal end having a second outer diameter that is larger than the first outer diameter; and an extended portion that extends from the distal end to the proximal end, the extended portion having an outer diameter that gradually increases from the first outer diameter of the distal end to the second outer diameter of the proximal end, wherein the distal end, proximal end, and extended portion of the hypo-tube are sized to prepare a peripheral artery balloon that mimics a varying diameter of a natural artery.

The hypo-tube of any preceding clause, further comprising an inner lumen that extends from the distal end to the proximal end of the hypo-tube.

The hypo-tube of any preceding clause, wherein the inner lumen has a substantially constant outer diameter that is the same size from the distal end to the proximal end of the hypo-tube.

The hypo-tube of any preceding clause, wherein the inner lumen has an outer diameter that gradually increases in size from the distal end to the proximal end of the hypo-tube.

The hypo-tube of any preceding clause, wherein a thickness of the hypo-tube is defined between an outer diameter of the inner lumen and the outer diameter of the hypo-tube.

The hypo-tube of any preceding clause, wherein a thickness at the distal end of the hypo-tube is the same as a thickness at the proximal end of the hypo-tube.

The hypo-tube of any preceding clause, wherein a thickness (α) at the distal end of the hypo-tube is less than a thickness (β) at the proximal end of the hypo-tube.

The hypo-tube of any preceding clause, wherein a thickness (α) at the distal end of the hypo-tube is 0.5 mm and a thickness (β) at the proximal end of the hypo-tube is 1 mm.

The hypo-tube of any preceding clause, wherein the thickness gradually increases from the distal end of the hypo-tube to the proximal end of the hypo-tube.

The hypo-tube of any preceding clause, wherein an outer diameter of the inner lumen at the proximal end of the hypo-tube is 0.1 mm larger than the outer diameter of the inner lumen at the distal end of the hypo-tube.

The hypo-tube of any preceding clause, wherein the extended portion includes at least two transitions in an outer diameter of the hypo-tube between the distal end and the proximal end of the hypo-tube.

The hypo-tube of any preceding clause, wherein a first transition of the extended portion has an outer diameter that is 0.5 mm larger than the first outer diameter at the distal end of the hypo-tube.

The hypo-tube of any preceding clause, wherein a second transition of the extended portion has an outer diameter that is 0.5 mm larger than the outer diameter at the first transition of the extended portion.

The hypo-tube of any preceding clause, wherein the second outer diameter at the proximal end of the hypo-tube is 0.5 mm larger than the outer diameter at the second transition of the extended portion.

The hypo-tube of any preceding clause, wherein (i) the first outer diameter at the distal end of the hypo-tube is 2.5 mm, (ii) the outer diameter at the first transition of the extended portion is 3 mm, (iii) the outer diameter at the second transition of the extended portion is 3.5 mm, and (iv) the second outer diameter at the proximal end of the hypo-tube is 4 mm.

The hypo-tube of any preceding clause, wherein the outer diameter of the extended portion of the hypo-tube that gradually increases from the first outer diameter of the distal end to the second outer diameter of the proximal end comprises a continuous slope.

The hypo-tube of any preceding clause, wherein the outer diameter of the extended portion of the hypo-tube is free of step changes in diameter.

A method of remodeling arteries of a patient after an atherectomy, the method comprising (a) providing a single peripheral artery balloon, the single peripheral artery balloon having an outer diameter that varies from a distal end to a proximal end of the balloon in a manner that mimics a varying diameter of a natural artery; (b) inserting the single peripheral artery balloon into a damaged artery of the patient; (c) expanding the single peripheral artery balloon to remodel the damaged artery; (d) deflating the single peripheral artery balloon; and (e) removing the single peripheral artery balloon from the patient.

The method of any preceding clause, wherein the single peripheral artery balloon is configured to remodel the popliteal artery of the leg without any other balloons being inserted into the popliteal artery of the leg.

The method of any preceding clause, wherein the single peripheral artery balloon is configured to remodel the superficial femoral artery (SFA) of the leg without any other balloons being inserted into the SFA of the leg.

A method of treating arteries of a patient, the method comprising (a) providing a single peripheral artery balloon, the single peripheral artery balloon having an outer diameter that varies from a distal end to a proximal end of the balloon in a manner that mimics a varying diameter of a natural artery; (b) inserting the single peripheral artery balloon into an artery of the patient; (c) expanding the single peripheral artery balloon to treat the artery; (d) deflating the single peripheral artery balloon; and (e) removing the single peripheral artery balloon from the patient.

The method of any preceding clause, wherein the single peripheral artery balloon is configured to treat the popliteal artery of the leg without any other balloons being inserted into the popliteal artery of the leg.

The method of any preceding clause, wherein the single peripheral artery balloon is configured to treat the superficial femoral artery (SFA) of the leg without any other balloons being inserted into the SFA of the leg.

The method of any preceding clause, wherein the method comprises a balloon angioplasty.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

1. A peripheral artery balloon comprising:
a distal end having a first outer diameter;
a proximal end having a second outer diameter that is larger than the first outer diameter; and
an extended portion that extends from the distal end to the proximal end, the extended portion having an outer diameter that gradually increases from the first outer diameter of the distal end to the second outer diameter of the proximal end,
wherein the distal end, proximal end, and extended portion of the peripheral artery balloon are sized to mimic a varying diameter of a natural artery.

2. The peripheral artery balloon of claim 1, further comprising an inner lumen that extends from the distal end to the proximal end of the balloon.

3. The peripheral artery balloon of claim 2, wherein the inner lumen includes at least one port for infusion and/or deflation of the balloon.

4. The peripheral artery balloon of claim 1, wherein the first outer diameter at the distal end of the balloon is 2.5 mm.

5. The peripheral artery balloon of claim 1, wherein the second outer diameter at the proximal end of the balloon is 4 mm.

6. The peripheral artery balloon of claim 1, wherein the extended portion includes at least two transitions in an outer diameter of the balloon between the distal end and the proximal end of the balloon.

7. The peripheral artery balloon of claim 6, wherein a first transition of the extended portion has an outer diameter that is 0.5 mm larger than the first outer diameter at the distal end of the balloon.

8. The peripheral artery balloon of claim 7, wherein a second transition of the extended portion has an outer diameter that is 0.5 mm larger than the outer diameter at the first transition of the extended portion.

9. The peripheral artery balloon of claim 8, wherein the second outer diameter at the proximal end of the balloon is 0.5 mm larger than the outer diameter at the second transition of the extended portion.

10. The peripheral artery balloon of claim 9, wherein (i) the first outer diameter at the distal end of the balloon is 2.5 mm, (ii) the outer diameter at the first transition of the extended portion is 3 mm, (iii) the outer diameter at the second transition of the extended portion is 3.5 mm, and (iv) the second outer diameter at the proximal end of the balloon is 4 mm.

11. The peripheral artery balloon of claim 1, wherein the balloon is one of (i) non-compliant, (ii) semi-complaint, or (iii) compliant.

12. The peripheral artery balloon of claim 1, wherein the natural artery is one of the popliteal artery and the superficial femoral artery (SFA).

13. The peripheral artery balloon of claim 1, wherein the first outer diameter of the distal end, the second outer diameter of the proximal end, and the gradually increasing outer diameter of the extended portion mimics the varying diameter of a natural artery in a 1:1 ratio.

14. A hypo-tube for preparing a peripheral artery balloon, the hypo-tube comprising:
a distal end having a first outer diameter;
a proximal end having a second outer diameter that is larger than the first outer diameter; and
an extended portion that extends from the distal end to the proximal end, the extended portion having an outer diameter that gradually increases from the first outer diameter of the distal end to the second outer diameter of the proximal end,
wherein the distal end, proximal end, and extended portion of the hypo-tube are sized to prepare a peripheral artery balloon that mimics a varying diameter of a natural artery.

15. The hypo-tube of claim 14, further comprising an inner lumen that extends from the distal end to the proximal end of the hypo-tube.

16. The hypo-tube of claim 15, wherein the inner lumen has a substantially constant outer diameter that is the same size from the distal end to the proximal end of the hypo-tube.

17. The hypo-tube of claim 15, wherein the inner lumen has an outer diameter that gradually increases in size from the distal end to the proximal end of the hypo-tube.

18. The hypo-tube of claim 15, wherein a thickness of the hypo-tube is defined between an outer diameter of the inner lumen and the outer diameter of the hypo-tube.

19. The hypo-tube of claim 18, wherein a thickness at the distal end of the hypo-tube is the same as a thickness at the proximal end of the hypo-tube.

20. The hypo-tube of claim 18, wherein a thickness (α) at the distal end of the hypo-tube is less than a thickness (β) at the proximal end of the hypo-tube.

21. The hypo-tube of claim 18, wherein a thickness (α) at the distal end of the hypo-tube is 0.5 mm and a thickness (β) at the proximal end of the hypo-tube is 1 mm.

22. The hypo-tube of claim 18, wherein the thickness gradually increases from the distal end of the hypo-tube to the proximal end of the hypo-tube.

23. The hypo-tube of claim 15, wherein an outer diameter of the inner lumen at the proximal end of the hypo-tube is 0.1 mm larger than the outer diameter of the inner lumen at the distal end of the hypo-tube.

24. The hypo-tube of claim 14, wherein the extended portion includes at least two transitions in an outer diameter of the hypo-tube between the distal end and the proximal end of the hypo-tube.

25. The hypo-tube of claim 24, wherein a first transition of the extended portion has an outer diameter that is 0.5 mm larger than the first outer diameter at the distal end of the hypo-tube.

26. The hypo-tube of claim 25, wherein a second transition of the extended portion has an outer diameter that is 0.5 mm larger than the outer diameter at the first transition of the extended portion.

27. The hypo-tube of claim 26, wherein the second outer diameter at the proximal end of the hypo-tube is 0.5 mm larger than the outer diameter at the second transition of the extended portion.

28. The hypo-tube of claim 26, wherein (i) the first outer diameter at the distal end of the hypo-tube is 2.5 mm, (ii) the outer diameter at the first transition of the extended portion is 3 mm, (iii) the outer diameter at the second transition of the extended portion is 3.5 mm, and (iv) the second outer diameter at the proximal end of the hypo-tube is 4 mm.

29. The hypo-tube of claim 14, wherein the outer diameter of the extended portion of the hypo-tube that gradually increases from the first outer diameter of the distal end to the second outer diameter of the proximal end comprises a continuous slope.

30. The hypo-tube of claim 29, wherein the outer diameter of the extended portion of the hypo-tube is free of step changes in diameter.

31. A method of remodeling arteries of a patient after an atherectomy, the method comprising:
a) providing a single peripheral artery balloon, the single peripheral artery balloon having an outer diameter that varies from a distal end to a proximal end of the balloon in a manner that mimics a varying diameter of a natural artery;
b) inserting the single peripheral artery balloon into a damaged artery of the patient;
c) expanding the single peripheral artery balloon to remodel the damaged artery;
d) deflating the single peripheral artery balloon; and
e) removing the single peripheral artery balloon from the patient.

32. The method of claim 31, wherein the single peripheral artery balloon is configured to remodel the popliteal artery of the leg without any other balloons being inserted into the popliteal artery of the leg.

33. The method of claim 31, wherein the single peripheral artery balloon is configured to remodel the superficial femoral artery (SFA) of the leg without any other balloons being inserted into the SFA of the leg.

34. A method of treating arteries of a patient, the method comprising:
a) providing a single peripheral artery balloon, the single peripheral artery balloon having an outer diameter that varies from a distal end to a proximal end of the balloon in a manner that mimics a varying diameter of a natural artery;
b) inserting the single peripheral artery balloon into an artery of the patient;
c) expanding the single peripheral artery balloon to treat the artery;
d) deflating the single peripheral artery balloon; and
e) removing the single peripheral artery balloon from the patient.

35. The method of claim 34, wherein the single peripheral artery balloon is configured to treat the popliteal artery of the leg without any other balloons being inserted into the popliteal artery of the leg.

36. The method of claim 34, wherein the single peripheral artery balloon is configured to treat the superficial femoral artery (SFA) of the leg without any other balloons being inserted into the SFA of the leg.

37. The method of claim 34, wherein the method comprises a balloon angioplasty.
